(19) United States
(12) Patent Application Publication
(10) Pub. No.: US 2005/0095200 A1
(43) Pub. Date: May 5, 2005

(54) Prokinetic drugs assistance to small intestine imaging

(76) Inventor: Moshe Schwarzberg, Ramat Gan (IL)

Correspondence Address: DANIEL J SWIRSKY, PO BOX 2345, BEIT SHEMESH, 99544, IL

(21) Appl. No.: 10/974,801

(22) Filed: Oct. 28, 2004

(30) Foreign Application Priority Data
Oct. 29, 2003 (IL) IL 158658

(51) Int. Cl. A61K 49/00; A61K 31/445; A61K 31/165

(52) U.S. Cl. 424/9.4; 514/317; 514/620

(57) ABSTRACT

The present invention discloses a novel method of increasing the efficacy of small intestine wall video-capsule imaging, by means of controlled administration of pro-kinetic medications. This cost effective method is adapted inter alia for in vivo imaging of the cavities in the digestive tract by means of pro-kinetic medication, comprising among other means administering an predetermined dose of a pro-kinetic drug to increase the forward push of the video-capsule and/or segmentary peristaltic contractions; and obtaining an image of the digestive tract wall, resulting in an increase percentage of visual information about the internal gastrointestinal cavity.

### CROSS-REFERENCE TO RELATED APPLICATIONS

This application claims priority under 35 U.S.C. 119 from Israel Patent Application No. 158,658, filed Oct. 29, 2003.

### FIELD OF THE INVENTION

The present invention generally relates to a method of imaging cavities in the digestive tract, and more particularly to a method of increasing the efficacy of small intestine wall video-capsule imaging, by means of controlled administration of pro-kinetic medications.

### BACKGROUND OF THE INVENTION

A number of devices effective for in vivo imaging and scanning of the small intestine are known in the literature. For example, researchers from Given Imaging Ltd., Israel registered a series of patents, such as U.S. Pat. No. 6,632,171, presenting a commercial ingestible video-capsule (hereinafter: ‘m2a’) utilized as a miniature wireless camera for color imaging of the small intestine walls, and particularly of those areas not accessible to enteroscopic imaging. The diagnostic yield obtained by use of such a video-capsule ranges from about 55% to 71%.

Since the transmitted pictures are of high quality, a resolution problem is ruled out, but it is possible that the failure to locate some of the pathologies is due to missed segments in the walls of the small intestine during passage of the video-capsule, so that part of the wall area is not imaged. Another substantial problem is the difficulty encountered by the capsule in crossing the antro-duodenal junction in cases of pyloric hypo motility.

U.S. Pat. Nos. 6,264,917 and 6,331,289 disclose a number of substances effective as a contrast agent in imaging of body cavities, particularly by means of ultra-sound scanning. These contrast agents, which are composed of a suspension of liquid soup and materials which contain or produce gas, bind to the target organs thus marking them and/or sharpening the resolution of the ultra-sound image. To date, no effective means for enhancing the video-capsule image are in use.

Video-capsule imaging of the small intestine with the available means is today the first-line diagnostic tool. A rise of 10% in the diagnostic yield would be liable to make such imaging the sole recommended diagnostic procedure. However, such means are not today readily accessible to physicians.

### SUMMARY OF THE INVENTION

The present invention therefore presents a novel and effective method for in vivo imaging of cavities in the digestive tract (e.g., small intestine) by use of pro-kinetic medications, comprising inter alia the steps of administering an appropriate dose of pro-kinetic medication so as to increase forward motion of the video-capsule and/or segmentary peristaltic contractions, and obtaining an image of the small intestine wall, resulting in an increase percentage of visual information about the internal gastrointestinal cavity.

In a preferred embodiment of the present invention, the said method is particularly suitable for detection of surface bleeding sites in the digestive tract, such as inflammations, ulcers and vascular malformations and/or for detection of surface bleeding sites in the small intestine wall.

In a general embodiment of the present invention, the pro-kinetic drug or combination of several such drugs is chosen from a wide range of active agents capable of increasing digestive system activity in general and stomach and small intestine activity in particular and/or accelerating transit of the imaging device through the intestine. These agents may be selected from one or more families, as detailed below.

In a preferred embodiment of the present invention, the pro-kinetic drug contains benzamides or their derivatives, and particularly derivatives of N-(3-hydroxy-4-piperidinyl) benzamides. These derivatives may be selected in a non limiting manner from the following agents: CIS-4-AMINO-5-CHLORO-N-(1-(4-FLUOROPHENOXY)PROPYL)3 METHOXY-4-PIPERIDINYL)-2 METHOXYBENZAMIDE, AMINO-4 CHLORO-5 N-(DIETHYLAMINO-2 ETHYL) METHOXY-2 BENZAMIDE, from compounds thereof, from their derivatives, and/or from compounds of the derivatives of such agents, administered to the subject orally, intravenously or otherwise. Preferably, the pro-kinetic drug is selected from commercially available Cisapride or Metoclopramide, or a combination thereof, in particular when Cisapride is administered to the subject in an oral dose of 20 to 30 mg. In another embodiment of the present invention, the pro-kinetic drug contains, among others, antibiotics of the macrolide family, including erythromycin and its derivatives.

In another preferred embodiment of the present invention, small intestine interior imaging is performed by means of endoscopy, antroscopy and/or laparoscopy. In another preferred embodiment of the present invention small intestine interior imaging is performed by means of a miniature wireless camera. In another preferred embodiment of the present invention small intestine interior imaging is performed by means of a video camera and/or miniature wireless video-capsule.

In another preferred embodiment of the present invention, the said method includes the following two steps: (a) administering 20 to 30 mg of Cisapride or Metoclopramide orally or intravenously, (b) obtaining an image of the digestive tract wall, such that effective visual information about the internal area or contracted segments (depressions) therein is presented.

In another preferred embodiment of the present invention, the said method includes the following three steps: (a) administering 20 to 30 mg of Cisapride or Metoclopramide orally or intravenously, (b) swallowing a camera or wireless miniature video-capsule, (c) imaging the internal wall of the digestive tract by camera and/or video and transmitting the picture thus received to the physician.

Concurrently or in addition, the present invention presents a novel and effective method for regulating the speed or motion regime of a miniature video-capsule in the patient's digestive tract by administering an appropriate dose of a pro-kinetic drug, so as to obtain a high diagnostic yield image of the small intestine wall.

Thus, in another preferred embodiment of the present invention the said method enables in vivo imaging of cavities in the digestive tract by use of pro-kinetic medications, including among other means, administering an appropriate dose of a pro-kinetic drug to increase the forward push of the video-capsule and/or segmentary peristaltic contractions, obtaining an image of the digestive tract wall, such that effective visual information about the internal area or contracted segments (depressions) therein is presented, wherein the transit of the video-capsule through the patient's digestive tract is regulated by administering an appropriate dose of a pro-kinetic drug.

In another preferred embodiment of the present invention a method is presented for passage of the video-capsule from the subject's stomach to small intestine via the duodenum by administering an appropriate dose of a pro-kinetic drug. This method includes the following three steps: (a) administering Cisapride or Metoclopramide orally or intravenously, (b) swallowing a camera or wireless miniature video-capsule, (c) imaging the internal wall of the digestive tract by camera and/or video and transmitting the picture thus received to the physician.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the details set forth above present only the basic details of the method and the modes of its implementation.

The present invention presents, as stated, a method for in vivo imaging of cavities in the digestive tract by use of pro-kinetic medications, and particularly a method for improving the quality of small intestine video-capsule imaging by use of pro-kinetic medications. This novel method is effective in increasing the range of motion (i.e., the photo angle) of the camera head; thus the imaged area of the intestinal walls will be increased and a significant improvement in the diagnostic yield generated by means of the video-capsule will be achieved. Although the small intestine is hereby discussed in a non-limiting manner, it is in the scope of the present invention to prove the aforementioned method in any portion of the gastrointernal tract.

Increase in the photo angle is achieved by altering the motion regime of the capsule. Two main forces act on the capsule in its transit through the intestine: (1) the push forwards; (2) the segmentary peristaltic contractions. Intensification of the segmentary contractions will create deeper contracted segments (depressions) and increase the sweep of the camera head during its stay in a depression. Pro-kinetic drugs are capable of accelerating the motility of materials in the digestive tract, normally resulting in a controlled increase in the push forwards and in the segmentary peristaltic contractions

Pro-kinetic drugs accelerate the passage of materials through the digestive tract, normally resulting in a controlled increase in the push forwards and in the segmentary peristaltic contractions. The action of these drugs is associated with the release mechanisms of intermediaries, which act on digestive tract activity, such as acetylcholine or dopamine or can act directly on the smooth muscle. Metoclopramide, which will be described below, and Domperidone are examples of dopamine antibodies. Metoclopramide and Cisapride, which will also be described below, increase release of acetylcholine. Bethanecol for example, binds directly to the muskarine receptor on the smooth muscle. Other medications with pro-kinetic properties include antibiotics of the Macrolide family, such as erythromycin, oxybutinin and ondanseteron hydrochloride. All of these substances will be denoted in the present invention, signally and jointly, as “pro-kinetic drugs”.

It is therefore proposed in the present invention to use pro-kinetic drugs of various types, as described above, for imaging of digestive tract cavities, and in particular for enhancing small intestine video-capsule imaging. According to the present invention, several different types of active agents may be used, including those containing various derivatives of benzamides, such as N-(3-hydroxy-4-piperidinyl)benzamides, which are effective pro-kinetic drugs.

An example of such drugs is Cisapride in which CIS-4-AMINO-5-CHLORO-N-(1-(4-FLUOROPHENOXY) PROPYL)3 METHOXY-4-PIPERIDINYL)-2 METHOXYBENZAMIDE MONOHYDRATE is the active agent. These drugs are marketed under the brand names Cisapride or Prepulsid and are currently manufactured by Janssen Pharmaceutical Inc and others. This family of agents will be denoted in the present invention simply as Cisapride. Concurrently and/or in addition, drugs in which the active agent is AMINO-4 CHLORO-5 N-(DIETHYLAMINO-2 ETHYL) METHOXY-2 BENZAMIDE are effective pro-kinetic drugs in their charged form and as de-chlorohydrate. This derivative and its family are commercially marketed under the brand names Metoclopramide and Reglan. This family of agents will be denoted in the present invention simply as Metoclopramide.

While the odds of capturing a polyp projecting into the bowel cavity are relatively high, effective imaging of the interior wall of the intestine or depressions therein is more difficult. The pro-kinetic drugs referred to above increase the detection rate of surface bleeding pathologies in the interior of the digestive tract, such as inflammations, ulcers and vascular malformations. These pathologies are not necessarily picked up by the video-capsule camera as it passes along, if the camera head is not pointing directly at the affected area. Increased motility thus creates deep segmentary depressions and tosses the capsule trapped in them about. Such conditions will enable the camera head to move in many more directions, or angles, and greatly enhances the quality of the image transmitted to the physician.

The effect of Cisapride on the motion of the video-capsule in the intestine may be gathered from its effect, at various dosages, on the parameters of solid or liquid material motion, e.g., chyme or barium. Based on studies of patients with irritable bowel syndrome and on radiography of the small intestine containing barium meal one can conclude in the present invention that the intensity of segmentary contraction may be insufficient at a daily dose of about 10 to 15 mg of Cisapride or an about single 20 mg dose of Metoclopramide. The term ‘about’ refers hereinafter to standard deviations and accuracy of up to 20% of the defined value. Manometric measurement showed that the contraction amplitude in the jejunum increased by about 43% upon intravenous injection of about 10 mg Cisapride, which is equivalent to about 25 mg orally, e.g., bio-availability equals about 40%. The contraction frequency also increased, which means there were more depressions, i.e., sites creating a wide imaging arc per unit length of intestine. A significant increase of amplitude of over about 18.4% in mmHg in the duodenum and jejunum in healthy subjects was obtained upon administration of about 30 mg/day of Cisapride by mouth (10×3). There was no change in the number of waves or in the wavelength. Peak plasma concentration of Cisapride is reached after about 1 to 2 hours and the half-life is about 10 hours (manufacturer's data). The stomach emptying time is not affected by intake of about 10 mg Cisapride by mouth (t_{1/2} equals about 78 min), but a dosage of about 20 mg by mouth reduces the stomach-emptying time of liquid food in healthy subjects: t_{1/2} is decreased by about 17.8%, down from 73 to about 60 minutes. On the basis of peak plasma concentration and stomach-emptying time data, one may conclude that a short interval between swallowing the vide-capsule and the Cisapride is desirable. The anticipated percentage of side effects is about 25%, but only in about 16% of cases is abdominal discomfort to be expected and abdominal pain only in about 4%. These data suggest the possibility of materially increasing the yield of the video-capsule.

Vigorous motility prevents or at least decreases the chances of “getting stuck” at junctions, e.g., at the antro-duodenal junction due to pyloric motility disorder, which then requires the physician to use an endoscope to push the capsule forward through the digestive tract. An about 10 mg dose of Cisapride by mouth decreases the transit time of solid food in healthy subjects by about 20%. Due to rapid filming time, e.g., two frames per second, even doubling the speed of motility does not result in loss of surface coverage capacity and the ratio between filming time and surface unit remains adequate. This ratio, which expresses the surface coverage capacity of the camera, is about 4.6 frames per cm², without accelerating the motility. It stands to reason that the video-capsule, e.g., a solid body measuring 26×11 mm, will behave similarly to solid food, given that the mean transmit time from mouth to large intestine is almost identical, e.g., 302 minutes as against 310 minutes respectively.

The contra-indication to the administration of Cisapride in cases of bleeding from the digestive tract refers to protracted use, at the accepted dosage of about 30 to 80 mg/day. There is no reference to one-time treatment of an adult, at a low dosage, of about 20 to 30 mg/day. A Medline cross-correlation, carried out throughout its length, between all causes of bleeding from the digestive tract (12,155 publications) and all publications containing some reference to Cisapride (1,709 in all) found not a single report of bleeding from the digestive tract caused by Cisapride. On the contrary, Cisapride is mentioned as a medication for treating bleeding from the upper digestive tract, secondary to gastro-esophageal reflex. It stands to reason that in planned experiments the authors refrained from prescribing this medication to patients suffering from intestinal bleeding, but the fact that there is not a single report of bleeding damage as a result of this medication, not even when taken inadvertently, through ignorance of an intestinal bleeding problem, or mistakenly due to physician-patient misunderstanding, suggests that the contra-indication is a precaution adopted by the manufacturer prior to marketing. Apparently, the possibility of one-time use at low dosage by an adult did not come up at the time of application for approval by the health authorities. In addition, it should be borne in mind that even if bleeding is caused by Cisapride intake, it will probably be minimal due to the low dosage, since the intensity of intestine contraction it is relatively low compared with the contraction caused by a full treatment dosage. The video-capsule has a significant advantage in locating an active bleeding lesion; the capsule reaches the spot from the section above the lesion, so that there is a strong contrast between the appearance of the healthy section of the intestine and the bleeding section. A colonoscope progresses through an intestine section covered with blood and therefore has more difficulty in locating the bleeding spot.

Metoclopramide, a pro-kinetic drug as described above, serves as another embodiment of the present invention. Metoclopramide has been presented in the literature as a dopamine d2 antagonist possibly able to prevent stomach bleeding caused by stimulation of dopamine receptors in the central nervous system. It is very efficient in treating stomach and small bowel motility disorders and could solve the problem of cross-over from the stomach to the duodenum. This medication could also improve, at least partially, the motion and photo angles in the small intestine. Metoclopramide is distinguished by a combination of a pyloric sphincter relaxing effect concurrently with intensification of the tonus and amplitude in the antrum. Metoclopramide has a high safety profile, even in intravenous injections at a dosage of about 10 mg as well as orally in a dosage of about 20 mg. Its absorption when taken orally is rapid and good, with a mean bio-availability equals about 77%, and a half-life time of about 5 hours. This promises efficacy as “pro-kinetic engine” until the video-capsule reaches the ileocecal valve, since the time taken to reach this point is identical to the mean transit time, about 302 minutes of the capsule from ingestion down to the colon.

Since the graph of Metoclopramide plasma concentration at various times after oral administration is linear, when tested at an oral dose of 10 mg, the peristaltic wave velocity speed or regularity of video-capsule progress through the digestive tract can be controlled by adjusting the dosage. The adjustment of speed of capsule motion through the small intestine depends also on the time interval between swallowing the tablets and swallowing the video-capsule. The speed of capsule progress, as well as the amplitude of contraction of the small intestine in its distal and relatively narrow part, may be reduced by increasing the interval between the times of ingestion. In order to achieve this, the capsule is to be ingested when the medication reaches its peak plasma concentration, e.g., about 56 minutes and less particularly, from about 30 to 75 min. The diameter of the intestine at the ileum is approximately equal to the length of the longitudinal axis of the capsule. The advantage gained by increasing the range of photo angles is diminished as the intestine diameter grows smaller, and vice versa, which means that pro-kinetic medication is particularly efficacious in large cavities.

A Medline cross-correlation, carried out throughout its length, between all the causes of bleeding in the digestive tract (12,155 publications) and all publications in which Metoclopramide is mentioned (5,347 publications) revealed not a single description of bleeding damage consequent to the use of this medication. On the contrary, Metoclopramide is mentioned as a possible treatment for esophageal variceal bleeding under portal hypertension, due to its ability to increase the low esophageal pressure while at the same time reducing the intravascular variceal pressure.

The present invention demonstrates for the first time that intensifying the segmentary contractions of the small intestine by means of pro-kinetic drugs significantly increases the range of motion, i.e., the photo angle, of the camera head. As a result the imaged area of the intestinal walls is increased and a significant improvement in the diagnostic yield generated by means of the video-capsule is achieved.

The present invention also demonstrates for the first time the surprising effectiveness of the method presented herein-above in imaging digestive tract cavities by use of pro-kinetic medication, and in more detail, in enhancing small intestine video-capsule imaging quality, by means of controlled administration of pro-kinetic drugs, such as be benzamides and its derivatives, and in particular an oral dose of about 25 to 30 mg Cisapride, with ingestion interval of 0 to about 15 minutes, or a full single dose of Metoclopramide.

The present invention also relates to imaging of the small intestine and in particular of the proximal half of the small intestine which has a large diameter relative to the distal half. It is therefore important to increase the angle and range of motion of the camera head over this part of the intestine. This increase is achieved if the video-capsule reaches the duodenum at approximately the same time as the pro-kinetic enhancing medication reaches optimal plasma concentration, normally peak concentration, and in the case of Cisapride or Metoclopramide about 60 to 90 minutes after ingestion. This time period varies between patients and depends on state of health (healthy or with gastroparesis), dosage, manner of administration (oral or parenteral), the state of aggregation of the substance taken (solid-semi-solid, liquid), the intake time-gap (medication relative to the taken substance) etc.

Considerable reliable data and experience have accumulated over more than 30 years of use of Metoclopramide and 19 years of use of Cisapride, during which clinical trials were conducted to establish the time needed to reach peak plasma concentration. Thus for example, intravenous administration of 8 mg Cisapride or 10 mg of Metoclopramide, 3 minutes after intake of barium, was observed to result in the barium being emptied from the stomach after 60 minutes in 85% and 93.5% of the subjects, respectively. In another experiment amongst diabetics suffering from gastroparesis who received semi-solid tagged substance, it was found that the mean half-life of stomach emptying was brought down from 110 to 67 minutes following intake of 30 mg Metoclopramide per day (3×10). In a group with similar characteristics, administration of 10 mg of Metoclopramide or 5 mg Cisapride intravenously showed lack of efficiency in emptying indigestible radio-opaque markers during the first hour of emptying. Oral intake of 15 mg Metoclopramide intake 30 minutes prior to the intake of tagged food reduced the food emptying half-life from the stomach in healthy subjects to 71.7 minutes as against 59.6 minutes in patients suffering from reflux esophagitis. A 20 mg dose of Cisapride by mouth reduces the food-emptying half-life of liquid food in healthy subjects by 17.8%, down from 73 to 60 minutes. It is known from the literature that the video-capsule, for example, commercially available m2a stays in the stomach for 55 minutes on average. From this it may be inferred that an oral dose of up to 15 mg will not significantly accelerate the motion of the video-capsule in the stomach. At the pharmaco-dynamically desirable dosage range, according to the present invention, i.e. a dose of about 25 to 30 mg, the transit time through the stomach will be significantly decreased. This time period depends on the intake time gap between the medication and the capsule.

Another aspect of the present invention, enabling for the first time comparison of the capsule movement against the background of the intestine walls, without operation of the video-capsule head camera, may be demonstrated by an experiment in which the patient ingests the pro-kinetic medication 10 minutes prior to the intake of contrast material. After another 30 minutes the capsule is swallowed. The capsule will reach the duodenum some 70 minutes from the intake of the medication. The intake time gap of 30 minutes between the contrast material and the capsule is intended to prevent the capsule from being dragged along in the liquid waves.

It is acknowledged that the video-capsule should move along the intestine solely by means of the peristaltic movement, wherein the capsule is detected as a pen-like shape comparing a rounded movement.

1. A method for in vivo imaging of the cavities in the digestive tract by means of pro-kinetic medication, comprising among other means administering an predetermined dose of a pro-kinetic drug to increase the forward push of the video-capsule and/or segmentary peristaltic contractions; and obtaining an image of the digestive tract wall, such that effective visual information about the internal area or contracted segments (depressions) therein is obtained.

2. The method according to claim 1, particularly adapted for detection of surface bleeding sites in the digestive tract wall, such as inflammations, ulcers and vascular malformations.

3. The method according to claim 1, particularly adapted for detection of surface bleeding sites in the small intestine wall.

4. The method according to claim 1, wherein the pro-kinetic medication contains benzamides or their derivatives.

5. The method according to claim 4, wherein the pro-kinetic medication contains among others derivatives of N-(3-hydroxy-4-piperidinyl) benzamides.

6. The method according to claim 4, wherein the pro-kinetic medication contains among others derivatives of cis-4-amino-5-chloro-n-(1-(4-fluorophenoxy) propyl)-3-methoxy-4-piperidinyl)-methoxybenzamide.

7. The method according to claim 4, wherein the pro-kinetic medication contains among others derivatives of 4-amino-5-chloro-n-(diethylamino-2 ethyl) methoxy-2-benzamide.

8. The method according to claim 4, wherein the pro-kinetic medication is selected from the commercial products Cisapride or Metoclopramide.

9. The method according to claim 8, wherein the medication is administered to the subject by mouth at a dose of about 20 to 30 mg.

10. The method according to claim 1, wherein the pro-kinetic medication contains amongst others an antibiotic of the macrolide family.

11. The method according to claim 10, wherein the antibiotic is aritromatzine.

12. The method according to claim 1, whereby imaging of the digestive tract interior is performed by means of endoscopy, antroscopy and/or laparoscopy.

13. The method according to claim 1, whereby imaging of the digestive tract interior is performed by means of a miniature wireless camera.

14. The method according to claim 13, whereby imaging of the digestive tract interior is performed by means of a video camera or miniature wireless video capsule.

15. The method according to claim 12, comprising
administering about 20 to 30 mg of Cisapride or Metoclopramide orally or intravenously; and,
obtaining an image of the digestive tract wall, such that effective visual information about the internal area or depressions in the digestive tract wall is obtained.

16. The method according to claim 13, comprising
administering about 20 to 30 mg of Cisapride or Metoclopramide orally or intravenously;
swallowing a camera or wireless miniature video-capsule; and,
(c) imaging the internal wall of the digestive tract by camera and/or video and transmitting the picture thus received to the physician.

17. The method according to claim 14, comprising
administering about 20 to 30 mg of Cisapride or Metoclopramide orally or intravenously;
swallowing a camera or wireless miniature video-capsule; and,
(c) imaging the internal wall of the digestive tract by camera and/or video and transmitting the picture thus received to the physician.

18. A method enabling control of the motion regime of a miniature video capsule through the digestive tract of the patient, so as to obtain an image of the digestive tract interior by administering an appropriate dose of a pro-kinetic drug.

19. A method for in vivo imaging of the cavities in the digestive tract by means of pro-kinetic medication according to claim 18, including among other means, administering an appropriate dose of a pro-kinetic drug to increase the forward push of the video-capsule and/or segmentary peristaltic contractions, obtaining an image of the digestive tract wall, such that effective visual information about the internal area or contracted segments (depressions) therein is obtained, wherein the motion regime of the miniature video capsule through the digestive tract of the patient is controlled by administration of an appropriate dose of a pro-kinetic drug.

20. A method enabling passage of the video-capsule from the subject's stomach to small intestine via the duodenum by administering an appropriate dose of a pro-kinetic drug, comprising
administering about 20 to 30 mg of Cisapride or Metoclopramide orally or intravenously;
swallowing a camera or wireless miniature video-capsule;
(c) imaging the internal wall of the digestive tract by camera and/or video and transmitting the picture thus received to the physician.
